(19) United States
(12) Patent Application Publication
(10) Pub. No.: US 2023/0010696 A1
(43) Pub. Date: Jan. 12, 2023

(54) ABSORPTION ENHANCING WEARABLE DEVICE

(71) Applicant: Anantha K. Pradeep, Piedmont, CA (US)

(72) Inventor: Anantha K. Pradeep, Piedmont, CA (US)

(21) Appl. No.: 17/369,863

(22) Filed: Jul. 7, 2021

(51) Int. Cl. A61N 1/32; A61N 1/04; A61N 1/36

(52) U.S. Cl. CPC A61N 1/327; A61N 1/0456; A61N 1/36034

(57) ABSTRACT

Provided are mechanisms and processes for enhancing absorption of therapeutic agents into the skin. Therapeutic agents may include lotions, creams, ointments, and solutions. Electrodes are used to apply a voltage to create an electric field to enhance penetration of the agent through the epidermal layer of the skin. The voltage applied may vary between 5V and 36V depending on the skin type or type of substance. The electrodes may be integrated into a wearable device such as a glove, sleeve, mask, bandage, etc.

### BACKGROUND

Various substances, when applied to the skin, can be used as therapeutic agents to moisturize the skin by locking in moisture, and/or deliver other substances such as a serum, active ingredient, and/or medication to be absorbed by the skin. The effectiveness of the therapeutic agents depends on factors such as the formulation of the substance applied and the amount of the substance absorbed by the skin. For instance, if a lotion is designed to deliver an active ingredient to the skin by absorption into the skin, the effectiveness of the active ingredient can be reduced if the lotion doesn't penetrate the skin effectively.

Various methods can be used to increase absorption of therapeutic agents to the skin, but these methods may have limited applicability depending on the type and condition of the skin being treated. For instance, for people with sensitive skin or a skin condition such as eczema, exfoliating or using a dermaroller before applying a substance may be irritating to the skin. In addition, if increased quantities of a substance are applied to encourage increased absorption, the substance may feel heavy or thick when applied and may use much more of the substance than is absorbed, thereby wasting a portion of the substance and making the treatment more expensive.

Consequently, it is desirable to improve existing methods and technologies to further enhance absorption of therapeutic agents into the skin.

### SUMMARY

Provided are various mechanisms and processes relating to a wearable device designed to improve penetration of a substance into the skin.

In one aspect, a wearable device includes electrodes including a first positive electrode and a first negative electrode. The first positive electrode and the first negative electrode have different orientations on the wearable device. The wearable device also include a membrane impermeable to oil and moisture substantially covering the surfaces of the wearable device. The membrane is configured to come into contact with a substance applied to the surface of the skin. The first positive electrode and the first negative electrode are configured to apply a voltage between 5V and 36V to create an electric field through the membrane and through the substance applied to the surface of the skin. The voltage and electric field can be configured to enhance penetration of the substance through the epidermal layer of the skin.

These and other embodiments are described further below with reference to the figures.

### BRIEF DESCRIPTION OF THE DRAWINGS

FIG. 1 illustrates an example of an electrical stimulation system that can be used with various implementations of a wearable device to improve penetration of a substance into skin.

FIG. 2 illustrates one configuration of a wearable device designed to improve penetration of a substance into skin.

FIGS. 3A-G illustrate various configurations of a wearable device designed to improve penetration of a substance into skin.

FIG. 4A illustrates one configuration of wearable device as a glove that is designed to improve penetration of a substance into skin.

FIG. 4B illustrates one configuration of wearable device as a sleeve that is designed to improve penetration of a substance into skin.

FIGS. 5A-C illustrate examples of a wearable device that is secured in place with an adhesive.

FIG. 6 is one example of a process for improving penetration of a substance into the skin at a selected location using electrode pads.

FIG. 7 is another example of a process for improving penetration of a substance into the skin at selected locations using electrode pads.

### DETAILED DESCRIPTION OF EXAMPLE EMBODIMENTS

In the following description, numerous specific details are set forth in order to provide a thorough understanding of the presented concepts. The presented concepts may be practiced without some or all of these specific details. In other instances, well known process operations have not been described in detail so as to not unnecessarily obscure the described concepts. While some concepts will be described in conjunction with the specific embodiments, it will be understood that these embodiments are not intended to be limiting.

### Introduction

Various substances, when applied to the skin, are used as therapeutic agents, such as moisturizers, serums, and medications. Moisturizers can take the form of lotions, creams, or ointments, which include varying relative amounts of water to keep the skin moist, and oily substances called occlusives, which are oily substances that block the water from evaporating from the skin. Moisturizers may also include other substances such as humectants, emollients, vitamins, menthol, lactic acid, etc. depending on the formulation.

The substances may be used as therapeutic agents that moisturize the skin by locking in moisture, and/or deliver other substances such as a serum, active ingredient, and/or medication to be absorbed by the skin. The effectiveness of the substances as therapeutic agents depends on factors such as the formulation of the substance applied and the amount of the substance absorbed by the skin. For instance, if a lotion is designed to deliver an active ingredient to the skin by absorption into the skin, the effectiveness of the active ingredient can be reduced if the lotion does not penetrate the skin effectively.

Various methods can be used to increase the application and absorption of therapeutic agents to the skin, such as preparing the skin by exfoliating, applying the agent when the skin is damp, applying steam to the skin, applying increased quantities of the agent to the desired area, using a dermaroller to create small channels in the skin to deliver the agent deeper into the skin, and/or choosing formulations that include a delivery system like small spheres that deliver active ingredients in layers or phases.

Despite the various methods that can be used to increase absorption of the therapeutic agents to the skin, these methods may have limited applicability depending on the type and condition of the skin being treated. For instance, for people with sensitive skin or a skin condition such as eczema, exfoliating or using a dermaroller may be irritating to the skin, and may even worsen the condition of the skin. In addition, the application of increased quantities of a substance to encourage increased absorption may be uncomfortable on the skin if it feels heavy or thick when applied. Furthermore, in order to encourage greater absorption by applying greater quantities of a substance may result in using much more of the substance than is absorbed, thereby wasting a portion of the substance, and making the treatment more expensive. Because the various methods previously used to encourage absorption have limited applicability, additional ways to increase absorption of a substance into the skin are desirable.

Accordingly, various implementations of a wearable device and associated methods are described herein to enhance absorption of substances such as therapeutic agents into the skin using electrodes that generate an electrical field that encourages deeper penetration of the substances into the skin than would occur without the application of the electrical field.

### System Examples

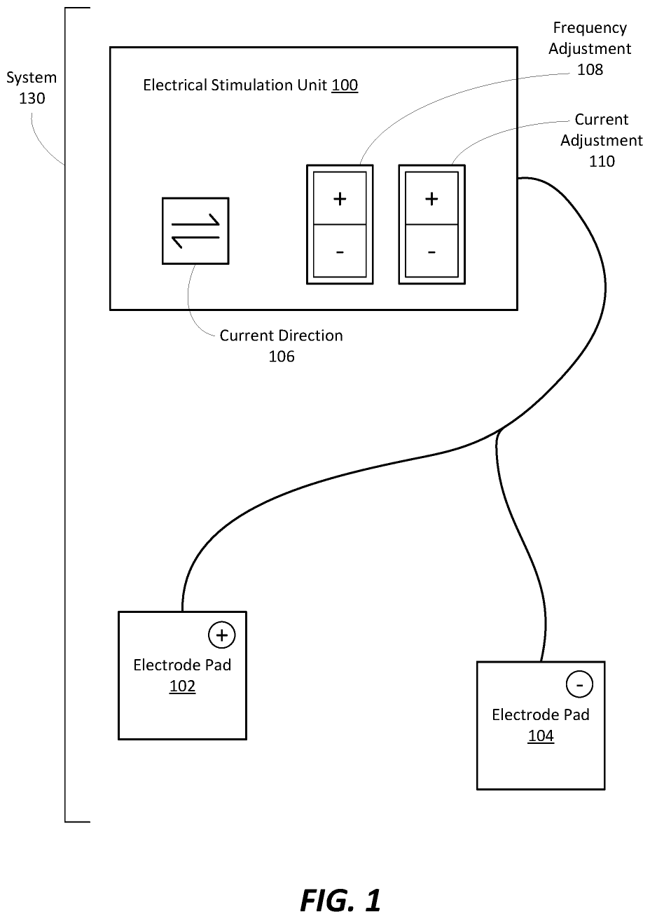

According to various embodiments, a wearable device is used to improve penetration of a substance into the skin with the use of an electrical stimulation system. With reference to FIG. 1, shown is one example of an electrical stimulation system that can be used as part of a wearable device designed to improve penetration of a substance into skin. Electrical stimulation system 130 includes an electrical stimulation unit 100 and electrode pads 102 and 104. According to various embodiments, the electrode pads 102 and 104 are skin surface electrodes designed to provide electrical stimulation to the skin and/or nerves.

In the present example, electrode pad 102 includes a positive electrode and a membrane that substantially covers the surface of the positive electrode and is impermeable to oil and moisture. Specifically, the membrane is designed to come into contact with a substance applied to the surface of the skin. Furthermore, electrode pad 102 includes a negative electrode and membrane that substantially covers the surface of the negative electrode and is impermeable to oil and moisture. Similarly, the membrane covering the negative electrode is designed to come into contact with a substance applied to the surface of the skin.

According to various embodiments, electrical stimulation unit 100 provides an electrical current to electrode pads 102 and 104, such that an electrical field is formed between the two electrode pads when applied to the surface of the skin. In particular, when electrode pads 102 and 104 are each placed on the surface of the skin, and a current is applied to the electrodes through electrical stimulation unit 100, electrical impulses are delivered through the skin from one electrode to the other. Accordingly, an electric field is created between the two electrodes that passes through the membranes and skin separating the two electrodes.

In the present example, electrical stimulation unit 100 includes controls that allow the adjustment of the current, voltage, and frequency of the electrical signals delivered to the electrodes depending on factors such as the desired intensity of treatment, and/or the user's skin type. As shown, frequency adjustment 108, which can be implemented as button(s), dial(s), or the like, allows adjustment of the number of electrical pulses sent per second. Furthermore, current adjustment 110, which can also be implemented as button(s), dial(s), or the like, allows adjustment of the intensity of the electrical pulses that are delivered through the electrode pads. In addition, current direction toggle 106, which can be implemented as a button, switch, etc., can be used to reverse the current so that the current flows from electrode pad 102 to electrode pad 104, or from electrode pad 104 to 102.

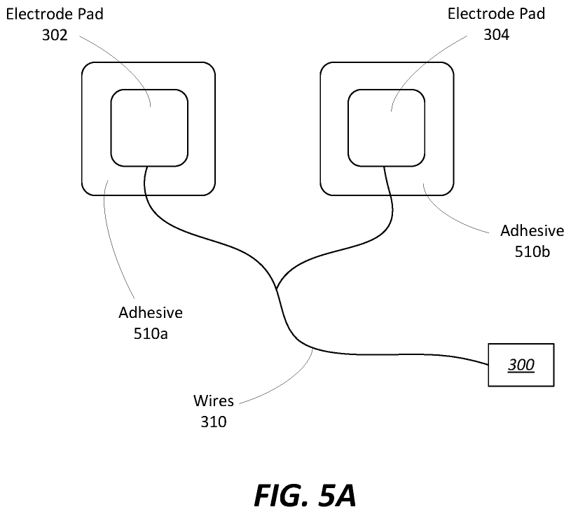

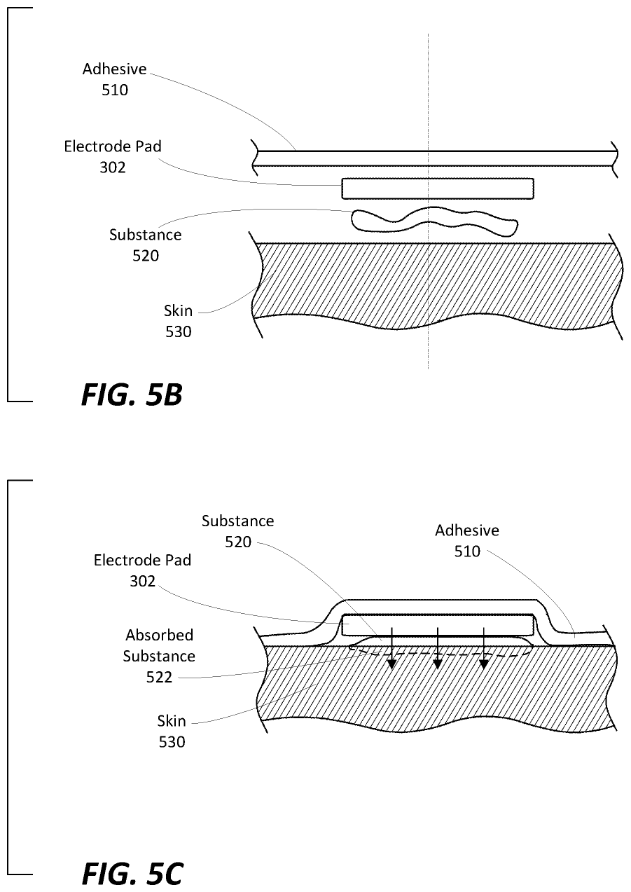

According to various embodiments described herein, system 130 is integrated into a wearable device to enhance penetration of a substance into the skin. In particular, as described in more detail below with regard to FIGS. 5B-C, when a substance is placed between the skin's surface and an electrode pad of system 130, penetration of the substance into the skin is enhanced when a voltage creating an electrical field is applied to the skin through the electrode pad. Although various voltages can be applied depending on the desired level of treatment, a range between around 5V and 36V can be applied through the electrodes, the membranes, and through the substance applied to the surface of the skin. In particular examples, the voltage is configured to vary between 10V and 15V to enhance penetration of the substance through the epidermal layer of the skin. However, various voltages and electric fields can be applied depending on the particular characteristics of a user's skin and what electric field achieves a desirable amount of penetration of a substance into the skin.

Although the present example includes two electrodes 102 and 104, system 130 can include any number of electrodes depending on the desired application. For instance, one type of unit that can be used with the wearable device is a transcutaneous electrical nerve stimulation (TENS) unit that has two sets of electrodes, each set having a positive and negative electrode. For such a TENS unit, the electrodes can be fastened to four locations on the skin. Similarly, additional electrodes may be included in the system, depending on the desired application.

In various implementations, if a TENS unit or similar device is incorporated into the wearable device, the intensity of electrical stimulation, frequency (i.e. electrical pulses per second), and duration (i.e. duration that the current enters the skin during each electrical pulse) can be adjusted depending on factors such as the desired level of penetration of the substance into the skin, the type of skin, and the comfort of the user. In some examples, if a TENS unit or similar device is also being used for pain relief, along with delivering a substance into the skin, the frequency can range from high frequency (HP) pulses from 80-120 cycles per second for acute pain to low frequency pulses from 1-20 cycles per second for chronic pain. Other frequency ranges can also be used depending on the desired treatment for the user.

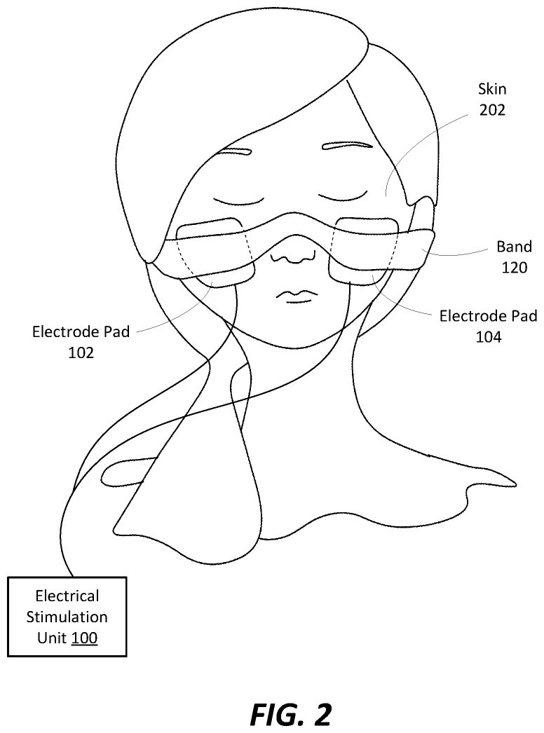

With reference to FIG. 2, shown is one configuration of a wearable device designed to improve penetration of a substance into skin. In this example, the wearable device is a facial covering that includes electrode pads 102 and 104 held in place by band 120 and connected to electrical stimulation unit 100, as described with regard to FIG. 1. As shown, the wearable device is applied to the face of a user. Electrode pads 102 and 104 may be oriented in different directions or may face different directions.

According to various embodiments, a substance is placed between the user's skin and electrode pads 102 and 104, and held in place with the help of band 120. When the electrical stimulation unit 100 delivers a current to the electrodes, an electrical field is formed between electrode pads 102 and 104. For instance, if the electrical signals are flowing from electrode 102 to 104 through the membrane of electrode 102 and through the user's skin towards electrode 104, the electrical field will encourage the substance to penetrate the skin by interacting with the substance to pull it further into the skin along the path of the electrical field.

According to various embodiments, the substance applied between the skin and the electrode can be a therapeutic agent, such as a moisturizer, serum, medication, or a combination of these agents, or the like. As mentioned previously, moisturizers can take the form of lotions, creams, or ointments, which include varying relative amounts of water to keep the skin moist, and oily substances called occlusives, which are oily substances that block the water from evaporating from the skin. Moisturizers may also include other substances such as humectants, emollients, vitamins, menthol, lactic acid, etc. depending on the formulation.

The substance may be used as a therapeutic agent by moisturizing the skin by locking in moisture, and/or delivering other substances such as a serum, active ingredient, and/or medication to be absorbed by the skin. The effectiveness of the substances as therapeutic agents depends on factors such as the formulation of the substance applied and the amount of the substance absorbed by the skin. Accordingly, to encourage greater absorption and/or penetration of the substance into the skin, the formulation of the substance applied in conjunction with use of the wearable devices described herein may include ingredients that encourage the substance to flow with the electrical field into the skin. For instance, the substance can be an electrically charged cream, lotion, or ointment that interacts with the electrical field to pull the substance further along the path of the electrical field than if no electrical field were applied.

In the present example, the substance is pulled more deeply into the skin when an electrical current is applied to electrodes 102 and 104, thereby enhancing the penetration of the substance into the skin. If the current is reversed so that the electrical current flows from electrode 104 to electrode 102, the substance applied between electrode 104 and the user's skin is then absorbed further as the substance is pulled more deeply into the skin as it follows the path of the electrical field applied.

According to various embodiments, the electrode pads 102 and 104 are held in place by a band 120, which can take on various forms depending on the desired application. For instance, the band 120 can include a stretchable material such as elastic or stretchy fabric that holds the pads in place. In some examples, band 120 can be a continuous material that stretches to fit the user. In other examples, band 120 can include one or more attachment mechanisms such as a hook and loop fastener (e.g. Velcro), snaps, ties, or other fasteners. Furthermore, depending on the application, the band can be flexible or rigid/semi-rigid. Examples of flexible bands 120 may include fabric (stretchy or non-stretch), latex, vinyl, or any other suitable material that can keep the electrodes in place as desired. Examples of rigid or semi-rigid bands 120 may include plastic or other materials that are shaped to hold the electrodes in place, such as a mask, or a series of panels that can be secured to the user.

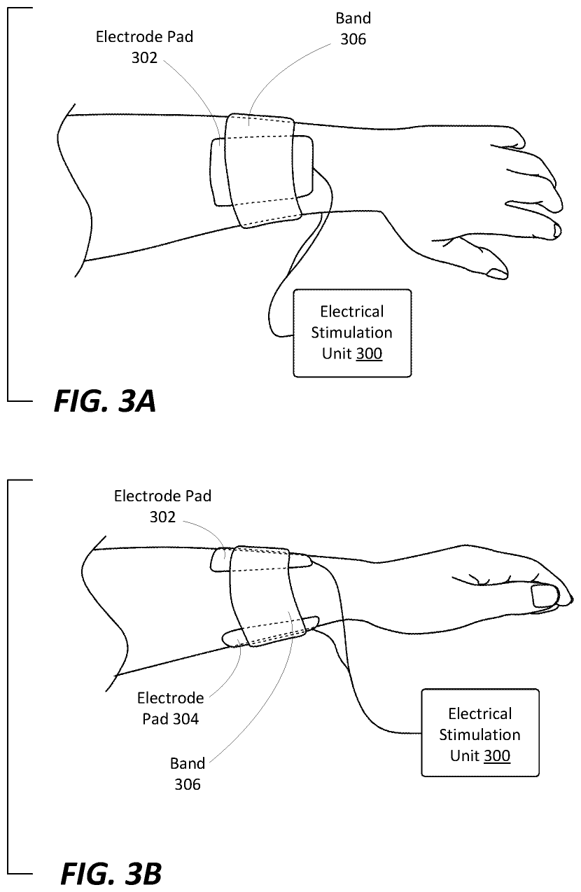

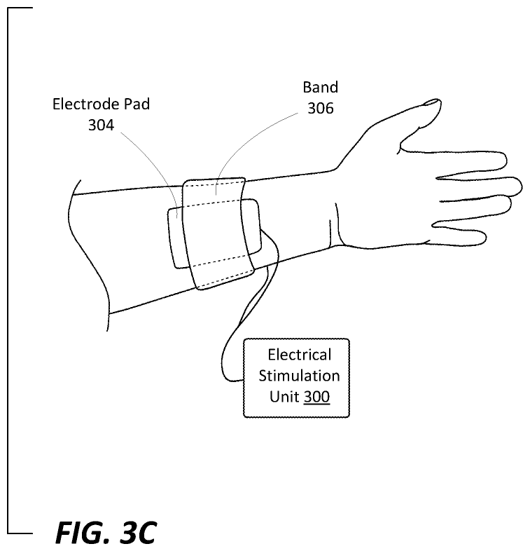

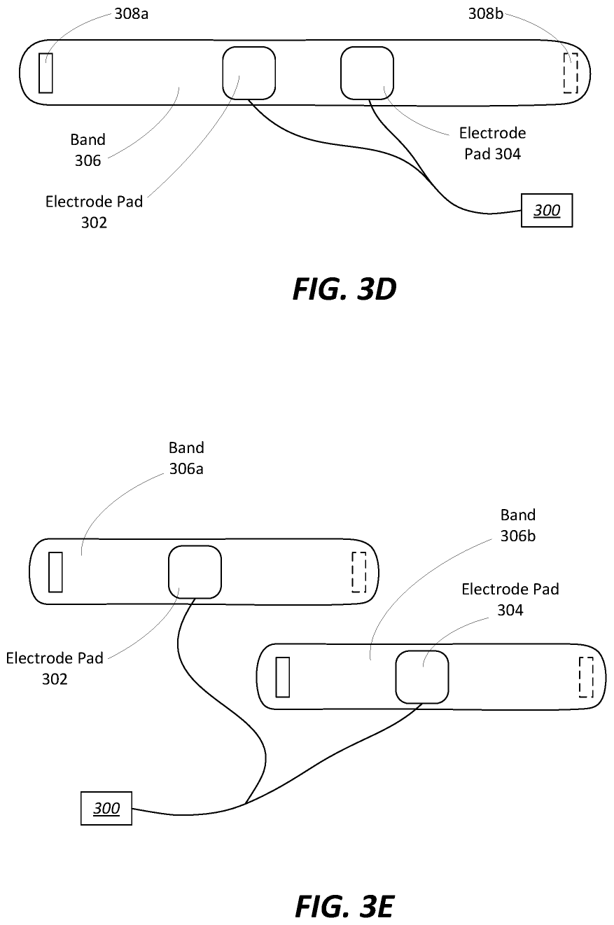

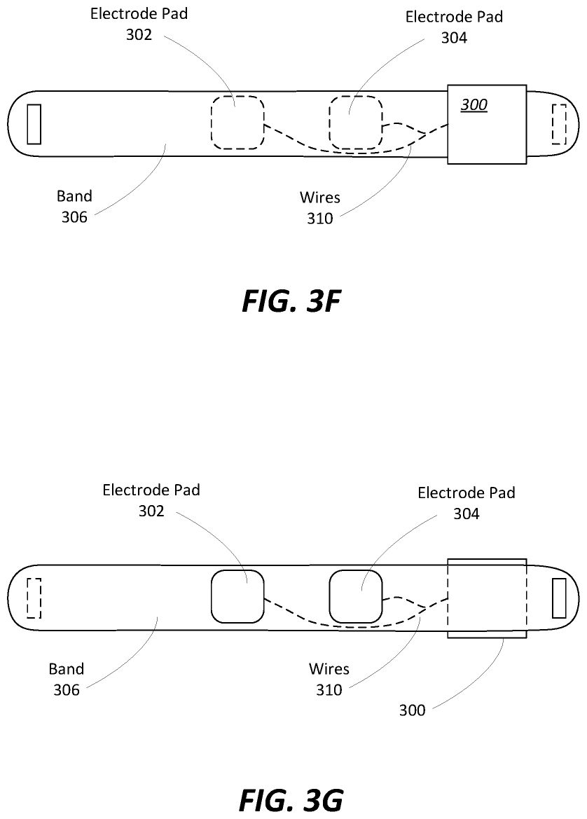

Although the illustration provided in conjunction with the present example shows that the electrodes extend beyond the width of band 120, it should be noted that any width and/or shape of band 120 can be used and any shape or size of electrode pads 102 and 104 can be used depending on the desired application. Furthermore, although the illustration of FIG. 2 shows a wearable device used to encourage penetration of a substance into two particular locations on the face, the electrodes can be placed at various locations depending on the desired application. For instance, electrode pads 102 and 104 can also be placed adjacent to each other on one side of the face, such as at two locations on one of the cheeks. Similarly, other configurations can also be used. In addition, wearable devices can also include configurations that improve penetration of a substance into skin at various locations on the body. In particular, FIGS. 3A-G illustrate various configurations of a wearable device designed to improve penetration of a substance into skin on other parts of the body.

With reference to FIGS. 3A-C, shown is one example of a wearable device used to improve penetration of a substance into skin on regions of the arm, as shown from different views. In the present example, the wearable device includes electrode pads 302 and 304 held in place by band 306 and connected to electrical stimulation unit 300. The electrode pads 302 and 304 are placed on generally opposite sides of the arm such that the electrical field created between the two electrodes passes into the surface of the skin in a direction that is generally perpendicular to the surface of the skin. By positioning the electrode pads 302 and 304 opposite each other, a substance placed between the skin and one or more of the electrode pads 302 and 304 may penetrate deeper into the surface of the skin than if the pads were positioned adjacent to each other or in another configuration.

In the present example, FIG. 3A shows a top view of a user's arm with a wearable device attached to the skin. From this view, electrode pad 302 is visible as it is positioned on the surface of the skin and secured with band 306. Electrode pad 302 is connected to electrical stimulation unit 300, and although not shown from this view, electrode pad 304 is also connected to electrical stimulation unit 300. Similar to the band described previously with regard to FIG. 2, band 306 can be made of various materials and may include different mechanisms to secure the electrodes to the skin at the desired locations, depending on the particular implementation.

With reference to FIG. 3B, shown is a side view of the user's arm with the wearable device worn described in conjunction with FIG. 3A. In particular, electrode pad 302 is positioned on a top surface of the arm and electrode pad 304 is positioned on a bottom surface of the arm such that the two electrodes are generally facing each other. In the present example, the electrode pads curve along the surface of the arm and create an electrical field between them when a current is applied by electrical stimulation unit 300.

Continuing with the present example, with reference to FIG. C, shown is a bottom view of the user's arm with the wearable device worn as described above with regard to FIGS. 3A-B. From this view, electrode pad 304 is visible and secured in place by band 306 and connected to electrical stimulation unit 300. Although a continuous band 306 is depicted in the figures, it should be noted that band 306 can be made of various materials and may include various attachment mechanisms that can be used to secure the electrode pads 302 and 304 to their desired locations. Furthermore, the width and thickness of band 306 can also vary depending on the application. For instance, although the band 306 is narrow enough to see the electrode pads 304 and 302 in the present example, band 306 can also be made wider to cover the electrode pads in other examples.

Additionally, although the electrode pads are shown in a particular size and shape, it should be recognized that the electrode pads can be made in different sizes and shapes depending on the desired treatment. For instance, larger pads could be used if it is desirable to treat a larger surface area for a particular duration. In another example, smaller pads could be used if it is desirable to target a particular location that is smaller. Alternatively, the substance applied between the electrode pads and the skin can be applied in the shape or area where absorption of the substance into the skin is most desirable.

FIGS. 3D-E illustrate various examples of wearable devices that can be used to improve penetration of a substance into the skin. With reference to FIG. 3D, shown is a wearable device that includes electrode pads 302 and 304 connected to an electrical stimulation device 300 and positioned on band 306. As described above, band 306 can be made of various materials depending on the desired application. In the present example, band 306 also includes attachment mechanisms 308a and 308b designed to engage each other to secure the band at a desired location on the body.

In the present example, electrode pads 302 and 304 can be separate from band 306, attached to band 306, or detachable from band 306. Specifically, if electrode pads are separate from band 306, band 306 can be used to hold the electrode pads in place once applied to desired locations on the skin. One advantage of keeping band 306 separate from the electrode pads is that the electrode pads can be positioned in a range of positions and distances away from each other. If electrode pads are attached to band 306, the distance between the two electrodes may be somewhat fixed depending on the amount of stretch allowed by band 306. Alternatively, the electrode pads can be slidably attached to band 306, such that the electrodes can be slid along the length of the band 306 to the desired locations. If the electrode pads are detachable from band 306, they can be removed for cleaning or replacement, and or repositioned along band 306. For instance, the electrode pads may be removably attached to band 306 with a hook and loop (e.g. Velcro) fastening mechanism or other suitable fastening mechanism.

With reference to FIG. 3E, shown is another example of a wearable device that includes electrode pads 302 and 304 connected to an electrical stimulation device 300 and positioned on bands 306a and 306b. Similar to the examples described previously, bands 306a and 306b can be made of various materials depending on the desired application. In the present example, bands 306a and 306b each include attachment mechanisms designed to secure the band at a desired location on the body. For instance, if a user wanted to position the electrode at two locations on the arm adjacent to each other, the bands could be positioned at these two locations with the desired distance between them. As with the previous example, electrode pads 302 and 304 can be separate from band 306, attached to band 306, or detachable from bands 306a and 306b, depending on the desired implementation.

FIGS. 3F-G depict another example of a wearable device that includes electrode pads 302 and 304 connected to an electrical stimulation device 300 and positioned on band 306. Specifically, FIGS. 3F and 3G show opposite sides of the wearable device with respect to band 306. In particular, with reference to FIG. 3F, a first side is shown where band 306 is visible and electrical stimulation unit 300 is visible and accessible. In this example, electrode pads 302 and 304 are positioned on the other side of band 306 and the wires connecting electrode pads 302 and 304 to unit 300 are hidden in this view.

Referring now to FIG. 3G, a second side is shown where electrode pads 302 and 304 are visibly positioned on band 306. As depicted, wires 310 are hidden within band 300, thereby making the wearable device more compact and contained. However, the wires may be visible on one side of the band in some examples if that configuration is more convenient for the desired implementation.

According to various embodiments, band 306 can be made of various materials depending on the desired application. In the present example, band 306 includes attachment mechanisms designed to secure the band at a desired location on the body. In addition, electrical stimulation unit 300, and electrode pads 302 and 304 are shown attached to band 306. Depending on the desired implementation, unit 300 and electrode pads may be attached to band 306 at fixed locations, slidably attached to band 306, or detachable from band 306, depending on the desired implementation. However, by embedding or otherwise connecting the wires 310 to the band 306, the wearable device can be used, transported, and/or stored as a single piece that is compact and easy to use. If the pads 302 and 304 and/or unit 300 are removably attached to wires 310, the wearable device can also be customizable and can accommodate replacement electrode pads and attachment of a different unit 300.

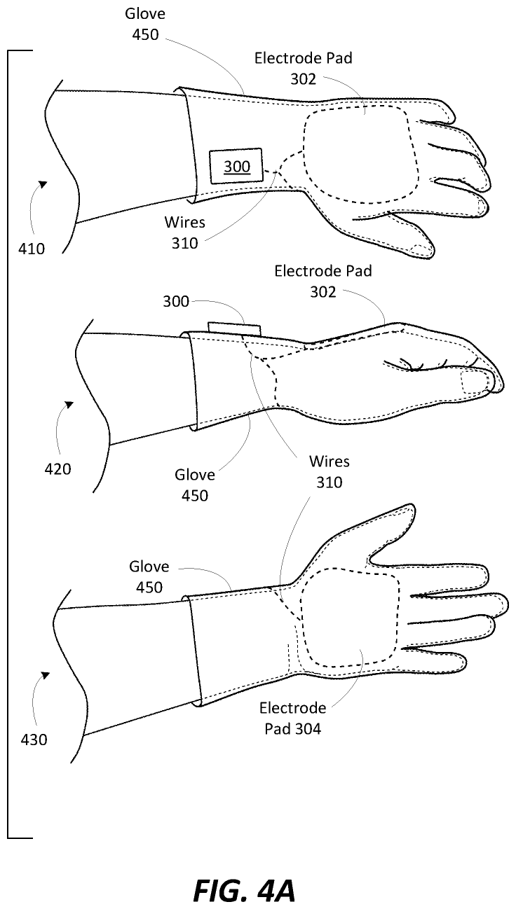

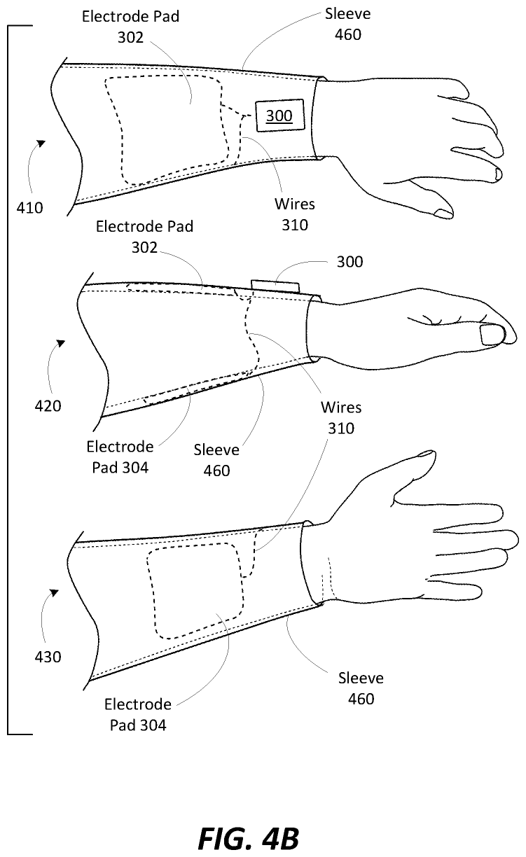

Although the previous examples show configurations of wearable devices with bands designed to keep electrodes in place at desired locations on the skin, other configurations of wearable devices can also be used to improve penetration of a substance into the skin. In particular, FIGS. 4A, 4B, and 5A-C each show alternative configurations of wearable devices that can also be used to encourage further penetration of a substance into the skin with the help of an applied electrical field.

With reference to FIG. 4A, shown is one configuration of wearable device as a glove that is designed to improve penetration of a substance into skin. In particular, glove 450 is shown from a top view 410, side view 420, and bottom view 430. In the present example, the wearable device is a glove 450 that includes electrode pads 302 and 304 and electrical stimulation unit 300. As shown in this example, the electrode pads 302 and 304 are placed on generally opposite sides of the hand such that the electrical field created between the two electrodes passes into the surface of the skin in a direction that is generally perpendicular to the surface of the skin. By positioning the electrode pads 302 and 304 opposite each other, a substance placed between the skin and one or more of the electrode pads 302 and 304 may be encouraged to penetrate deeper into the surface of the skin than if the pads were positioned adjacent to each other or in another configuration.

In the present example, the electrode pads 302 and 304 are positioned inside the glove 450 such that the material of the glove holds the electrode pads 302 and 304 against a user's hand when worn, as shown in views 410, 420, and 430. The electrode pads 302 and 304 are connected to electrical stimulation unit 300 by wires 310. As shown, wires 310 are not visible from the outside surface of the glove, and may either be integrated within the material of the glove such that most or all of the wires 310 do not come in contact with the skin or run along the inside surface of the glove such that the wires 310 may come in contact with the skin.

According to various examples, electrical stimulation unit 300 can be integrated into the glove and include controls that are accessible to from the top surface of the glove, as shown in views 410 and 420. In some implementations, electrical stimulation unit 300 can be disconnected from glove 450 for cleaning or replacement. Similarly, electrode pads 302 and 304 may be removable from glove 450 in some implementations. For instance, unit 300 and/or electrode pads 302 and 304 can be removably attached to glove 450 such as with a hook and loop mechanism (e.g. Velcro) or some other attachment mechanism. By integrating unit 300 and electrode pads 302 and 304 into glove 450 as a wearable device, glove 450 can conveniently and easily be worn by a user without the need to organize wires, devices, attachment mechanisms, etc.

In the present example, when the user is wearing glove 450, electrode pad 302 is positioned on a top surface of the hand and electrode pad 304 is positioned on a bottom surface of the hand such that the two electrodes are generally facing each other. In the present example, the electrode pads curve along the surfaces of the hand and create an electrical field between them when a current is applied by electrical stimulation unit 300. As described above with regard to various examples, when a substance is applied between the skin and the electrodes, penetration and/or absorption of the substance into the skin is enhanced when the electrical field is applied.

Continuing with the present example, although glove 450 is depicted in a particular style, it should be noted that glove 450 can be designed in various ways, such as without fingers, with openings in certain places, etc. In addition, glove 450 can be made of various materials and may include various attachment mechanisms that can be used to better secure the electrode pads 302 and 304 to their desired locations. For instance, glove 450 can be made of fabric, mesh, latex, vinyl, or any other suitable material or combination thereof. In some instances, the glove 450 can be designed to be reusable and washable, and in other instances, the glove can be designed to be disposable. Furthermore, glove 450 can be made in various sizes and/or can include one or more adjustment mechanisms to better fit a user and hold the electrodes in place. For instance, an adjustable strap could be positioned around the outside of the glove over the electrodes so that when tightened and secured either with elastic or a mechanism such as a buckle or Velcro, the electrodes are held in place more securely against the skin. Additionally, an adjustable strap can also be included near the location of unit 300 such that when tightened and secured, the strap holds unit 300 more securely in place without sagging or moving.

In the present example, although the electrode pads are shown in a particular size, shape, and location, it should be recognized that the electrode pads can be made in different sizes and shapes and be positioned at various locations depending on the desired treatment. For instance, larger or smaller pads could be used if it is desirable to treat a larger or smaller surface area for a particular duration. Alternatively, the substance applied between the electrode pads and the skin can be applied in the shape or area where absorption of the substance into the skin is most desirable. Also, the pads can be shaped to better fit the shape of the hand in some instances. Furthermore, if desired, the electrode pads can be positioned on the fingers or sides of the hands, depending on the desired treatment. Additionally, although unit 300 is shown to be located on the back side of the wrist area, unit 300 can be positioned at any convenient location, such as the back of the hand, inside of the wrist, inside of the hand, etc. depending on the desired implementation.

With reference to FIG. 4B, shown is a configuration of wearable device as a sleeve 460 that is designed to improve penetration of a substance into skin. In particular, sleeve 460 is shown from a top view 410, side view 420, and bottom view 430. In the present example, the wearable device is a sleeve 460 that includes electrode pads 302 and 304 and electrical stimulation unit 300. As shown in this example, the electrode pads 302 and 304 are placed on generally opposite sides of the arm such that the electrical field created between the two electrodes passes into the surface of the skin in a direction that is generally perpendicular to the surface of the skin. By positioning the electrode pads 302 and 304 opposite each other, a substance placed between the skin and one or more of the electrode pads 302 and 304 may be encouraged to penetrate deeper into the surface of the skin than if the pads were positioned adjacent to each other or in another configuration.

In the present example, the electrode pads 302 and 304 are positioned inside the sleeve such that the material of the sleeve holds the electrode pads 302 and 304 against the user's arm when worn, as shown in views 410, 420, and 430. The electrode pads 302 and 304 are connected to electrical stimulation unit 300 by wires 310. As shown, wires 310 are not visible from the outside surface of the sleeve, and may either be integrated within the material of the sleeve such that most or all of the wires 310 do not come in contact with the skin or run along the inside surface of the sleeve such that the wires 310 may come in contact with the skin.

According to various examples, electrical stimulation unit 300 can be integrated into the sleeve and include controls that are accessible to from the top surface of the sleeve, as shown in views 410 and 420. In some implementations, electrical stimulation unit 300 can be disconnected from sleeve 460 for cleaning or replacement. Similarly, electrode pads 302 and 304 may be removable from sleeve 460 in some implementations. For instance, unit 300 and/or electrode pads 302 and 304 can be removably attached to sleeve 460 such as with a hook and loop mechanism (e.g. Velcro) or some other attachment mechanism. By integrating unit 300 and electrode pads 302 and 304 into sleeve 460 as a wearable device, sleeve 460 can conveniently and easily be worn by a user without the need to organize wires, devices, attachment mechanisms, etc.

In the present example, when the user is wearing sleeve 460, electrode pad 302 is positioned on a top surface of the arm and electrode pad 304 is positioned on a bottom surface of the arm such that the two electrodes are generally facing each other. In the present example, the electrode pads curve along the surfaces of the arm and create an electrical field between them when a current is applied by electrical stimulation unit 300. As described above with regard to various examples, when a substance is applied between the skin and the electrodes, penetration and/or absorption of the substance into the skin is enhanced when the electrical field is applied.

Continuing with the present example, although sleeve 460 is depicted in a particular style, it should be noted that sleeve 460 can be designed in various ways, such as in various lengths, with openings in certain places, etc. In addition, sleeve 460 can be made of various materials and may include various attachment mechanisms that can be used to better secure the electrode pads 302 and 304 to their desired locations. For instance, sleeve 460 can be made of fabric, mesh, latex, vinyl, or any other suitable material or combination thereof. In some instances, sleeve 460 can be designed to be reusable and washable, and in other instances, sleeve 460 can be designed to be disposable. Furthermore, sleeve 460 can be made in various sizes and/or can include one or more adjustment mechanisms to better fit a user and hold the electrodes in place. For instance, an adjustable strap could be positioned around the outside of the sleeve over the electrodes so that when tightened and secured either with elastic or a mechanism such as a buckle or Velcro, the electrodes are held in place more securely against the skin. Additionally, an adjustable strap can also be included near the location of unit 300 such that when tightened and secured, the strap holds unit 300 more securely in place without sagging or moving.

In the present example, although the electrode pads are shown in a particular size, shape, and location, it should be recognized that the electrode pads can be made in different sizes and shapes and be positioned at various locations depending on the desired treatment. For instance, larger or smaller pads could be used if it is desirable to treat a larger or smaller surface area for a particular duration. Alternatively, the substance applied between the electrode pads and the skin can be applied in the shape or area where absorption of the substance into the skin is most desirable. Also, the pads can be shaped to better fit the shape of the arm in some instances. Furthermore, in some instances, the sleeve can be rotated on the arm so that electrode pads 302 and 304 are positioned on the sides of the arm, etc. Additionally, although unit 300 is shown to be located on the back side of the wrist area when the sleeve is worn, unit 300 can be positioned at any convenient location on the sleeve depending on the desired implementation.

FIGS. 5A-C illustrate examples of a wearable device that is secured in place with an adhesive. In some examples, this wearable device can be used alone or in conjunction with other materials that assist in placing and/or securing the electrode pads at the desired locations. For instance, the electrodes may be used in conjunction with a band, sleeve, glove, etc. depending on the desired implementation.

With reference to FIG. 5A, shown is an example of a wearable device that is secured in place with an adhesive. As with the previous examples described, this wearable device is designed to improve penetration of a substance into skin. In the present example, electrode pad 302 includes an adhesive region 510a that allows the electrode pad 302 to be secured in place at a desired location on the skin. Similarly, electrode pad 304 includes an adhesive region 510b that allows the electrode pad 304 to be secured in place at another desired location on the skin. As shown, electrode pads 302 and 304 are connected to electrical stimulation unit 300 by wires 310.

Although the adhesive pads 302 and 304 and adhesives 510a and 510b are shown in a particular shape and configuration in the present example, it should be recognized that the shape, configuration, and size can vary depending on the implementation. For instance, the adhesive may not surround the entire electrode in some implementations, such as if the horizontal length of the adhesive extends beyond the horizontal width of the electrode pad and the vertical height of the adhesive is equal to or less than the vertical height of the electrode pad. In addition, the type of adhesive can vary depending on the desired use. For instance, if the adhesive can be designed to be part of a reusable or disposable material and appropriate adhesives can be used accordingly.

Other configurations of the current example can also be implemented. For instance, in one configuration, unit 300 can be integrated into a band, sleeve, glove, etc. to allow the entire system to be wearable during treatment. In other configurations, unit 300 can be secured to a location on the skin with an adhesive (if the unit is small enough for this to be practical). In yet other configurations, unit 300 can be located directly above one of the electrodes and held in place by an adhesive and/or other mechanism such as a band, sleeve, glove, etc.

With reference to FIGS. 5B-C, shown are side cross-sectional views of a portion of a wearable device secured to the skin. In particular, FIG. 5B depicts a blow-apart diagram showing layers of an adhesive 510, electrode pad 302, and substance 520 relative to skin 530. As described above with regard to FIG. 5A, the size and shape of the adhesive 510 and electrode pad 302 can vary depending on the desired treatment. In addition, as also described above with regard to various embodiments, substance 520 can include a therapeutic agent, such as a moisturizer, serum, medication, or a combination of these agents, or the like. As mentioned previously, moisturizers can take the form of lotions, creams, or ointments, which include varying proportions of water and occlusives, and may also include other substances such as humectants, emollients, vitamins, menthol, lactic acid, etc. depending on the formulation. Furthermore, as also mentioned earlier, the formulation may also include ingredients that encourage the substance to flow with the electrical field into the skin to encourage greater absorption and/or penetration of the substance into the skin. For instance, the substance can be an electrically charged cream, lotion, or ointment that interacts with the electrical field to pull the substance further along the path of the electrical field than if no electrical field were applied.

With reference to FIG. 5C, shown is one example of a cross-sectional, side view showing layers of adhesive 510, electrode pad 302, and substance 520 when the wearable device is secured to skin 530. Specifically, the layers shown in FIG. 5B are secured in place such that adhesive 510 holds electrode pad 302 at a desired location on the skin. In the present example, substance 520 is applied between electrode pad 302 and skin 530. When a current is applied that flows from electrode pad 302 to another electrode pad 304 (not shown) that is located generally opposite electrode pad 302, substance 520 is pulled in the direction of the arrows into the skin such that absorbed substance 522 penetrates into skin 530. In the present example, substance 520 is pulled more deeply into the skin such that the amount of absorbed substance 522 is greater than if no electrical current were applied to electrode pad 302. If the direction of the current is reversed so that the electrical current flows from electrode 304 to electrode 302, the substance applied between electrode pad 520 and the user's skin is likely to migrate away from skin 530.

Although the present example depicts a device that includes an adhesive 510, it should be recognized that other configurations can also be used to improve penetration of a substance 520 into skin 530. For instance, other implementations, such as the ones described in previous examples can also be used to secure electrode pad 302 in place, such as with bands, sleeves, gloves, etc. Accordingly, the increased amount of absorbed substance 522 as depicted in the present example can also be achieved by using other configurations of wearable devices described previously, as long as an electrical field is applied that pulls the substance 520 into the skin.

Although the previous examples described above include particular configurations of electrodes and electrical stimulation units as wearable devices designed to improve penetration of a substance into skin, other configurations can also be implemented. For instance, it should be recognized that although examples above refer to a wearable device that includes two electrode pads, any number of electrode pads can be used. In some examples, if the configuration of a typical TENS unit is used, the wearable device would have two sets of electrode pads, each set having one positive electrode and one negative electrode. Similarly, other configurations can also be implemented whether as a custom configuration or as a configuration that implements an existing electrical stimulation system. For instance, if a system requires only one electrode, this single electrode can be integrated into the wearable device along with an electrical stimulation unit. In such examples, any number of single electrodes connected to an electrical stimulation unit may be included in a particular wearable device as desired. Alternatively, in an example using two electrodes, one of the electrodes can be located with the electrical stimulation unit to simplify the layout of the wearable device and to make the device more compact to use, store, and transport.

### Operating Examples

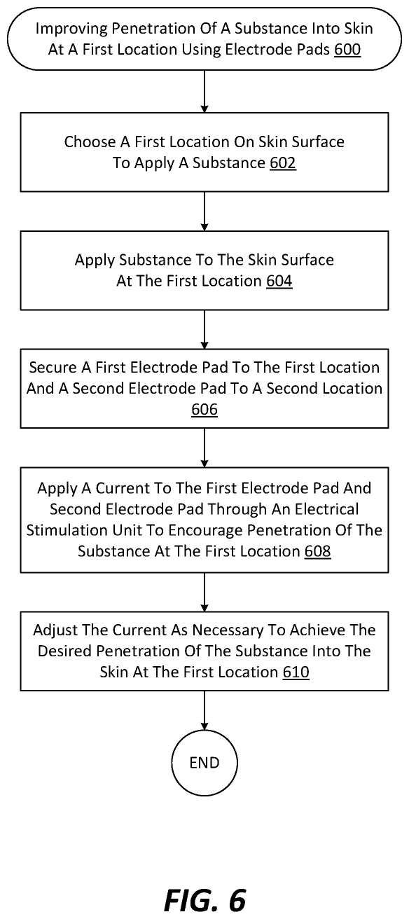

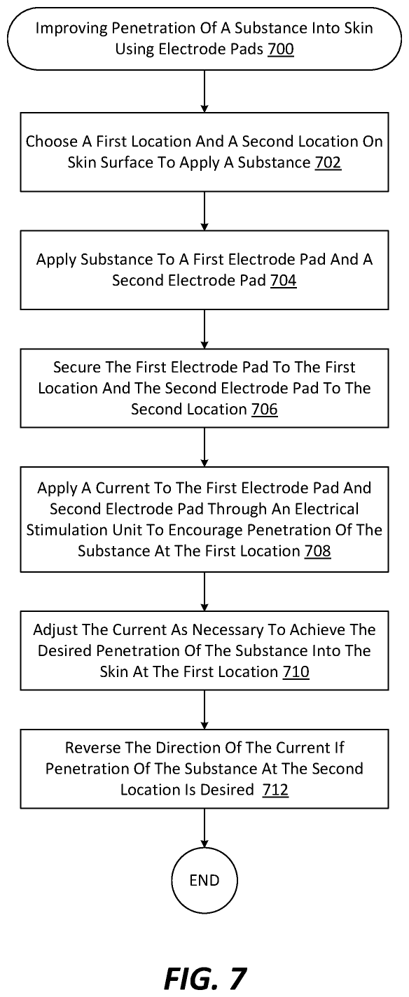

As described with regard to the examples above, a wearable device can be used to improve the penetration of a substance into the skin by applying an electrical field to the skin. These wearable devices can be used in a multitude of ways, depending on the desired treatment. FIGS. 6 and 7 include two examples of processes that can be used to improve penetration of a substance into the skin using these wearable devices. Although these particular processes are shown by example, it should be recognized that other processes can also be implemented using the wearable devices within the scope of this disclosure.

With reference to FIG. 6, one example of a process 600 is depicted for improving penetration of a substance into the skin at a selected location using electrode pads. According to various implementations, electrode pads, such as the ones described above with regard to various examples of wearable devices can be used to carry out this process 600. Other electrode pads can also be used with process 600 to achieve improved penetration of a substance into the skin.

In the present example, a first location to apply a substance on the skin is chosen at 602. As described above with regard to various examples, a substance can include a therapeutic agent, such as a moisturizer, serum, medication, or a combination of these agents, or the like. Furthermore, moisturizers can take the form of lotions, creams, or ointments, which include varying proportions of water and occlusives, and may also include other substances such as humectants, emollients, vitamins, menthol, lactic acid, etc. depending on the formulation. Additionally, the formulation may also include ingredients, such as an electrically charged cream, lotion, or ointment, or the like, that encourage the substance to flow with the electrical field into the skin to encourage greater absorption and/or penetration of the substance into the skin.

Next, at 604, the substance is applied to the skin surface at the first location. According to various embodiments, the first location can be chosen based on the desired therapy. For instance, some locations may encourage absorption of a medication more effectively and such locations may be chosen if deeper absorption of the medication is desired. In other instances, a location may be chosen because that area of the skin needs more absorption of moisture, etc.

Once the substance is applied at the first location, then at 606, a first electrode pad is secured to the first location and a second electrode pad is secured to a second location. In particular, the first electrode pad can be secured by any of a number of mechanisms, some of which are described above. Specifically, the first electrode pad can be secured using a band, sleeve, glove, adhesive, or other mechanism to keep the first electrode in place during treatment.

In the present example, the second location is selected depending on the location of the first location. For instance, if the main treatment location is at the first location, the second location can be selected depending on various factors, such as which locations encourage current flow between the first location and the second location when current is applied to the electrode pads. In a particular example, if the configuration depicted in FIGS. 3A-C is desirable to improve absorption of a substance at the location of electrode pad 302, then the location of electrode pad 304 can be selected depending on various factors. In the example shown, the location of electrode pad 304 is selected at a position generally opposite the location of electrode pad 302. In other examples, electrode pad can be placed adjacent to electrode pad 302. As shown in FIGS. 3A-C, the first and second electrode pads are secured at their locations with the assistance of band 306. As described previously, various mechanisms can be used to secure the first and second electrode pads in place.

Next, after the electrodes are secured in place, then at 608, a current is applied to the first electrode pad and second electrode pad through an electrical stimulation unit to encourage penetration of the substance at the first location. As described above with regard to various examples, the electrical stimulation unit can be used with the first and second electrode as part of a wearable device. In other examples, the electrical stimulation unit can be used with the first and second electrode regardless of whether the electrical stimulation unit and electrodes are integrated into a wearable device.

In the present example, at 610, the current can then be adjusted as necessary to achieve the desired penetration of the substance into the skin at the first location. For instance, the frequency and intensity of the current can be adjusted at the electrical stimulation unit, as well as the direction of the current flow. In particular, the current should flow from the first electrode pad to the second electrode pad in order to improve penetration of the substance into the skin at the first location. Additionally, adjustments can be made to the frequency and intensity of the current depending on factors such as the type of substance applied at the first location, the user's skin type, the desired amount of penetration of the substance, the user's comfort, etc.

Although the present example describes improving penetration of the substance only at the first location, penetration of the same or a different substance at the second location can also be achieved by applying the substance at the second location before securing the second electrode to the skin. If the direction of the current is reversed to flow from the second electrode pad to the first electrode pad, then penetration of the substance at the second location can also be improved.

With reference to FIG. 7, another example of a process 700 is depicted for improving penetration of a substance into the skin using electrode pads. In particular, electrode pads, such as the ones described above with regard to various examples of wearable devices can be used to carry out this process 700. Other electrode pads can also be used with process 700 to achieve improved penetration of a substance into the skin.

The process begins at 702, when a first location on the skin and a second location on the skin are chosen to apply the substance. As described above with regard to various examples, a substance can include a therapeutic agent, such as a moisturizer, serum, medication, or a combination of these agents, or the like. Furthermore, moisturizers can take the form of lotions, creams, or ointments, which include varying proportions of water and occlusives, and may also include other substances such as humectants, emollients, vitamins, menthol, lactic acid, etc. depending on the formulation. Additionally, the formulation may also include ingredients, such as an electrically charged cream, lotion, or ointment, or the like, that encourage the substance to flow with the electrical field into the skin to encourage greater absorption and/or penetration of the substance into the skin.

Next, at 704, the substance is applied to a first electrode pad and a second electrode pad. Once the substance is applied to each electrode pad, then at 706, the first electrode pad is secured to the first location and a second electrode pad is secured to the second location. In particular, the first and second electrode pads can be secured by any of a number of mechanisms, some of which are described above. Specifically, the electrode pads can be secured using a band(s), sleeve(s), glove(s), adhesive(s) or other mechanism(s) to keep the first electrode in place during treatment.

In the present example, the first and second locations are selected depending on various factors, such as the desired area(s) to be treated and locations that encourage current flow between the electrodes at the first location and the second location when current is applied to the electrode pads. In a particular example, if the configuration depicted in FIGS. 3A-C is desirable to improve absorption of a substance at the location of electrode pad 302, then the location of electrode pad 304 can be selected depending on various factors. In the example shown, the location of electrode pad 304 is selected at a position generally opposite the location of electrode pad 302. In other examples, electrode pad can be placed adjacent to electrode pad 302. As shown in FIGS. 3A-C, the first and second electrode pads are secured at their locations with the assistance of band 306. As described previously, various mechanisms can be used to secure the first and second electrode pads in place.

Next, after the electrodes are secured in place, then at 708, a current is applied to the first electrode pad and second electrode pad through an electrical stimulation unit to encourage penetration of the substance at the first location. As described above with regard to various examples, the electrical stimulation unit can be used with the first and second electrode as part of a wearable device. In other examples, the electrical stimulation unit can be used with the first and second electrode regardless of whether the electrical stimulation unit and electrodes are integrated into a wearable device.

In the present example, at 710, the current can then be adjusted as necessary to achieve the desired penetration of the substance into the skin at the first location. For instance, the frequency and intensity of the current, along with the direction of the current flow, can be adjusted at the electrical stimulation unit. In particular, the current should flow from the first electrode pad to the second electrode pad in order to improve penetration of the substance into the skin at the first location. Additionally, adjustments can be made to the frequency and intensity of the current depending on factors such as the type of substance applied at the first location, the user's skin type, the desired amount of penetration of the substance, the user's comfort, etc.

Next, at 712, the direction of the current is reversed if penetration of the substance at the second location is desired. Specifically, if the direction of the current is reversed to flow from the second electrode pad to the first electrode pad, then penetration of the substance at the second location can also be improved. Although the present example describes applying the same substance to both the first and second electrode pads, it is possible that in other examples, a different substance can be applied to each of the electrode pads if it is desirable to treat the two locations with different substances. Additionally, if improved penetration of the substance at only the first location is desired, then the substance may not need to be applied to the second electrode in some cases to achieve the desired result.

Although the foregoing concepts have been described in some detail for purposes of clarity of understanding, it will be apparent that certain changes and modifications may be practiced within the scope of the appended claims. It should be noted that there are many alternative ways of implementing the processes, systems, and apparatuses. Accordingly, the present embodiments are to be considered as illustrative and not restrictive.

What is claimed is:

1. A wearable device comprising:
a plurality of electrodes including a first positive electrode and a first negative electrode, the first positive electrode and the first negative electrode having different orientations on the wearable device;
a membrane impermeable to oil and moisture substantially covering the surfaces of the wearable device, the membrane configured to come into contact with a substance applied to the surface of the skin;
wherein the first positive electrode and the first negative electrode are configured to apply a voltage between 5V and 36V to create an electric field through the membrane and through the substance applied to the surface of the skin, wherein the electric field is configured to enhance penetration of the substance through the epidermal layer of the skin.

2. The wearable device of claim 1, wherein the substance is an electrically charged cream.

3. The wearable device of claim 1, wherein the substance is an electrically charged lotion.

4. The wearable device of claim 1, wherein the substance is an electrically charged ointment.

5. The wearable device of claim 1, wherein the wearable device is a facial covering

6. The wearable device of claim 1, wherein the wearable device is a glove.

7. The wearable device of claim 1, wherein the wearable device is a sleeve.

8. The wearable device of claim 1, wherein the voltage is configured to vary between 10V and 15V.

9. The wearable device of claim 1, wherein the voltage and electric field is user configurable based on skin type.

10. A method comprising:
choosing a first location on a skin surface to apply a substance, wherein the first location is a desired treatment location that would benefit from absorption of the substance into the epidermal layer, and applying the substance to the skin surface at the first location, wherein the substance is a therapeutic agent that is more effective when absorbed by the skin;
securing a first electrode pad to the first location and securing a second electrode pad to a second location, wherein the second location chosen based on the first location; and
applying a current to the first electrode pad and second electrode pad through an electrical stimulation unit, wherein the current flows from the first electrode pad to the second electrode pad through the skin to encourage penetration of the substance into the skin at the first location.

11. The method of claim 10, further comprising adjusting intensity of the current as necessary to achieve the desired penetration of the substance into the skin at the first location.

12. The method of claim 10, further comprising adjusting frequency of the current as necessary to achieve the desired penetration of the substance into the skin at the first location.

13. The method of claim 10, further comprising applying the substance to the skin surface at the second location before securing the second electrode pad to the second location.

14. The method of claim 13, further comprising reversing direction of the current to flow from the second electrode pad to the first electrode pad through the skin to encourage penetration of the substance into the skin at the second location.

15. The method of claim 10, wherein a voltage of between 10V and 15V is used to generate the current.

16. The method of claim 10, wherein the current is user configurable based on skin type.

17. A system comprising:
a first electrode pad that includes a first electrode substantially covered by a first membrane impermeable to oil and moisture and configured to come into contact with a substance applied to a skin surface, wherein the first electrode pad is integrated into a wearable device that is configured to be worn by a user during a skin treatment;
a second electrode pad that includes a second electrode substantially covered by a second membrane impermeable to oil and moisture and configured to come into contact with the substance, wherein the second electrode pad is integrated into the wearable device;
an electrical stimulation unit configured to create an electric field between the first electrode and second electrode through the first membrane, second membrane and through the substance applied to the skin, wherein the electric field is configured to enhance penetration of the substance through the epidermal layer of the skin when the user is wearing the wearable device and a current is applied to the first and second electrode, wherein the electrical stimulation unit is integrated into the wearable device.

18. The system of claim 17, wherein the wearable device is a facial covering, glove, sleeve, or band.

19. The system of claim 17, wherein the substance is electrically charged.

20. The system of claim 17, wherein a voltage between 5V and 36V is applied to create the electric field.
